(19) United States
(12) Patent Application Publication
(10) Pub. No.: US 2022/0287990 A1
(43) Pub. Date: Sep. 15, 2022

(54) TOPICAL JAK INHIBITOR COMBINATION COMPOSITIONS FOR TREATMENT OF INFLAMMATORY SKIN CONDITIONS

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Ori NOV, Tarum (IL); Ofer TOLEDANO, Kfar Saba (IL)

(73) Assignee: Sol-Gel Technologies Ltd., Ness Ziona (IL)

(21) Appl. No.: 17/629,424

(22) PCT Filed: Jul. 23, 2020
(86) PCT No.: PCT/IL2020/050825

§ 371 Date: Jan. 24, 2022

Related U.S. Application Data
(60) Provisional application No. 62/933,567, filed on Nov. 11, 2019.
(60) Provisional application No. 62/877,975, filed on Jul. 24, 2019.

(51) Int. Cl. A61K 31/05; A61K 9/00; A61K 31/327; A61K 31/519; A61K 31/203; A61K 31/192; A61K 31/4436; A61K 31/4709; A61K 31/65; A61K 31/7056; A61K 31/7048; A61K 9/06

(52) U.S. Cl. CPC A61K 31/05; A61K 9/0014; A61K 31/327; A61K 31/519; A61K 31/203; A61K 31/192; A61K 31/4436; A61K 31/4709; A61K 31/65; A61K 31/7056; A61K 31/7048; A61K 9/06

(57) ABSTRACT

Provided herein is a topical combination composition and a method of treatment of an inflammatory skin condition by administration of said composition comprising at least one Janus kinase inhibitor (JAK inhibitor) and at least one additional active agent elected from benzoyl peroxide (BPO), at least one retinoid, tapinarof, at least one antibiotic, at least one antiandrogen, at least one acaricide and combinations thereof and a carrier suitable for topical administration. The JAK inhibitor and the at least one additional active agent exhibit an additive or synergistic effect, allowing to reduce the amounts of active agents in the composition.

### FIELD OF THE INVENTION

The present invention, in some embodiments thereof, relates to pharmaceutical topical combination compositions for the treatment of inflammatory skin conditions, comprising a JAK inhibitor and at least one additional active agent.

The compositions of this invention are useful for the treatment, prevention or amelioration of skin conditions and exhibit additive and/or synergistic effects.

### BACKGROUND OF THE INVENTION

Janus kinase inhibitors, also known as JAK inhibitors or jakinibs (henceforth JAK inhibitors or JAKi), are a class of drugs interfering with the JAK-STAT signaling pathway by inhibiting at least one of the Janus kinase enzymes JAK1, JAK2, JAK3 or TYK2. Some JAK inhibitors inhibit all the above enzymes and are therefore named pan-JAK inhibitors.

JAK-STAT is an intracellular signaling pathway upon which many different pro-inflammatory signaling pathways converge (Damsky W. J Am Acad Dermatol. 2017 April; 76(4): 736-744).

A small number of JAK inhibitors are already marketed in the US as oral drugs for the treatment of conditions like rheumatoid arthritis, psoriatic arthritis, ulcerative colitis (Xelianz), myelofibrosis and polycythemia vera (Jakafi). No topical JAK inhibitor is presently marketed.

Inflammatory skin conditions include conditions like acne, rosacea, atopic dermatitis, psoriasis, flexural/inverse psoriasis, eczema, contact dermatitis, urticaria, dermatitis herpetiformis, lichen planus and seborrheic dermatitis.

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes. Although early colonisation with Propionibacterium acnes and family history might have important roles in the disease, exactly what triggers acne and how treatment affects the course of the disease remain unclear (Williams H. C. et al., The Lancet, Vol. 379. January 2012, pp. 361-372).

There is no ideal treatment for acne, although a suitable regimen for reducing lesions can be found for most patients. Good quality evidence on comparative effectiveness of common topical and systemic acne therapies is scarce. Topical therapies including benzoyl peroxide, retinoids, and antibiotics when used in combination usually improve control of mild to moderate acne. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective acne therapy and is used early in severe disease, although its use is limited by teratogenicity and other side-effects.

Rosacea is a chronic disease of inflammatory dermatitis that mainly affects the median part of the face and the eyelids of certain adults. It is characterized by telangiectatic erythema, dryness of the skin, papules and pustules. Typically, rosacea develops in adults from the ages of 30 to 50; it more frequently affects women, although the condition is generally more severe in men. Rosacea is a primarily vascular condition whose inflammatory stage lacks the cysts and comedones characteristic of common acne.

Factors that have been described as possibly contributing towards the development of rosacea include for example: parasites such as the Demodex folliculorum, bacteria such as Helicobacter pylori (a bacterium associated with gastrointestinal conditions), hormonal factors (such as endocrine factors), climatic and immunological factors, and so forth.

Rosacea develops in four stages over several years, in spasms aggravated by variations in temperature, alcohol, spices, exposure to sunlight and stress.

The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage (papular-pustular rosacea) with appearance of inflammatory papules and pustules, but without affecting the sebaceous follicles and thus with absence of cysts and comedones.

Stage 4: rhinophyma stage. This late phase essentially affects men. The patients have a bumpy, voluminous red nose with sebaceous hyperplasia and fibrous reordering of the connective tissue.

Typical treatments of rosacea include oral or topical administration of antibiotics such as tetracyclines, salicylic acid, anti-fungal agents, steroids, metronidazole, isotretinoin in severe cases, or anti-infectious agents such as azelaic acid.

US 20110052515 describes a topically applicable formulation for treating rosacea, comprising at least one avermectin compound and benzoyl peroxide (BPO, an anti-acne agent.

Montes et al. (Cutis, 32, 185-190 (1983)) disclosed the use of BPO dissolved in acetone gel formulation for the treatment of rosacea.

Flexural/Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the centre. This rare form of psoriasis accounts for 3-7% of people with psoriasis. A small Chinese study found that the average age of onset for inverse psoriasis is 28.9 years. Occasionally people with another subtype of psoriasis known as pustular psoriasis go on to develop inverse psoriasis. Recent guidelines from the National Psoriasis Foundation recommend the use of low to moderate strength corticosteroids for flare ups of this type of psoriasis and calcipotriene and either tacrolimus or pimecrolimus (e.g. Elidel) for treatment of inverse psoriasis in the long term.

There is a need for long-term treatment of inflammatory skin conditions, their symptoms and associated conditions in a safe and effective manner, by a patient-friendly topical treatment.

### SUMMARY OF THE INVENTION

This invention provides a topical combination composition comprising from about 0.1% w/w to about 3.0% w/w at least one JAK inhibitor and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about 0.1% w/w to about 3% w/w at least one antiandrogen, from about 0.5% to about 5% w/w at least one acaricide and combinations thereof and a carrier suitable for topical administration.

This invention provides a topical composition comprising from about 0.1% w/w to about 3.0% w/w at least one JAK inhibitor and optionally at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about 0.1% w/w to about 3% w/w at least one antiandrogen, from about 0.5% to about 5% w/w at least one acaricide and combinations thereof and a carrier suitable for topical administration.

The topical administration of the combination composition of this disclosure is patient-friendly and avoids systemic side-effects.

The above composition is useful for the treatment, prevention or alleviation of inflammatory skin conditions by topical administration to a subject in need thereof a therapeutically effective amount of the composition of this invention according to the disclosed regimen of administration.

### DETAILED DESCRIPTION OF THE INVENTION

This invention provides a novel approach for the topical treatment of inflammatory skin conditions, based on the understanding that these conditions have several different phases and that each phase can be treated with a different class of active agents.

It occurred to the present inventors, that treating an inflammatory skin condition with a composition combining a JAK inhibitor with one or more additional active agents from different active agent classes is more effective than treating the inflammatory skin conditions with the individual single drugs or with combinations of active agents from the same class, affording higher clearance rates and/or longer remission periods between clearance and reappearance of the inflammatory skin disease.

More particularly, this invention provides a combination composition comprising from about 0.1% w/w to about 3.0% w/w at least one JAK inhibitor and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen and combinations thereof and a carrier suitable for topical administration.

The above active agents belong to seven different active agent classes, and can be combined in active agents combinations of two, three or more active agents, suitable for the treatment of one or more inflammatory skin conditions selected from acne, rosacea, atopic dermatitis, psoriasis, flexural/inverse psoriasis, eczema, contact dermatitis, urticaria, dermatitis herpetiformis, lichen planus and seborrheic dermatitis.

Exemplary Active Agents' Combination Compositions (see also Tables 1 and 2 below):

A topical combination composition comprising from about 0.1% w/w to about 3.0% w/w at least one JAK inhibitor, from about 2% w/w to about 10% w/w benzoyl peroxide (BPO), from about 0.01% w/w to about 0.3% w/w at least one retinoid and a carrier suitable for topical administration in the treatment, prevention or alleviation of an inflammatory skin condition.

A topical combination composition comprising from about 0.1% w/w to about 3.0% w/w at least one JAK inhibitor, from about 2% w/w to about 10% w/w benzoyl peroxide (BPO) and a carrier suitable for topical administration in the treatment, prevention or alleviation of an inflammatory skin condition.

A topical combination composition comprising from about 0.1% w/w to about 3.0% w/w at least one JAK inhibitor, from about 0.01% w/w to about 0.3% w/w at least one retinoid and a carrier suitable for topical administration in the treatment, prevention or alleviation of an inflammatory skin condition.

A topical combination composition comprising from about 0.1% w/w to about 3.0% w/w at least one JAK inhibitor, from about 0.1% w/w to about 2% w/w tapinarof and a carrier suitable for topical administration, in the treatment, prevention or alleviation of an inflammatory skin condition.

A topical combination composition comprising from about 0.1% w/w to about 3.0% w/w at least one JAK inhibitor, from about 0.1% w/w to about 3.0% w/w at least one antibiotic, and a carrier suitable for topical administration in the treatment, prevention or alleviation of an inflammatory skin condition.

A topical combination composition comprising from about 0.1% w/w to about 3.0% w/w at least one JAK inhibitor, from about 0.1% w/w to about 3% w/w at least one antiandrogen, from about 0.5% to about 5% w/w at least one acaricide and a carrier suitable for topical administration in the treatment, prevention or alleviation of an inflammatory skin condition.

A topical combination composition comprising from about 0.1% w/w to about 3.0% w/w at least one JAK inhibitor, from about. 0.1% w/w to about 3% w/w at least one antiandrogen, from about 0.5% to about 5% w/w at least one acaricide from about 0.1% w/w to about 2% w/w at least one antibiotic, for topical administration in the treatment, prevention or alleviation of an inflammatory skin condition.

A topical combination composition comprising from about 0.1% w/w to about 3.0% w/w at least one JAK inhibitor, from about 2% w/w to about 10% w/w benzoyl peroxide (BPO), from about 0.1% w/w to about 2% w/w at least one antibiotic and a carrier suitable for topical administration in the treatment, prevention or alleviation of an inflammatory skin condition.

The at least one JAK inhibitor in the compositions of this invention is selected from a JAK1 inhibitor, a JAK2 inhibitor, a JAK3 inhibitor, a TYK2 inhibitor and combinations thereof

In some embodiments, there is provided a topical composition for the treatment, prevention or alleviation of an inflammatory skin condition, comprising from about 0.1% w/w to about 3.0% w/w at least one JAK inhibitor and at least one additional active agent selected from about 2% w/w to about 10% w/w BPO, from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration,

wherein said at least one JAK inhibitor is selected from tofacitinib, abrocitinib, ruxolitinib, delgocitinib, oclacitinib, baricitinib, peficitinib and combinations thereof,
wherein said inflammatory skin condition is selected from acne, rosacea, atopic dermatitis, psoriasis, flexural/inverse psoriasis, eczema, contact dermatitis, urticaria, dermatitis herpetiformis, lichen planus and seborrheic dermatitis,
wherein said at least one retinoid is selected from tretinoin, adapalene, tazarotene and combinations thereof,
wherein said at least one antibiotic is selected from ozenoxacin, minocycline, doxycycline, clindamycin, clarithromycin, erythromycin and combinations thereof, wherein said at least one antiandrogen is selected from clascoterone, cyproterone, cioteronel and combinations thereof and
wherein said acaricide is selected from ivermectin and permethrin.

In some embodiments, there is provided a topical composition for the treatment, prevention or alleviation of an inflammatory skin condition selected from acne and rosacea, comprising from about 0.1% w/w to about 3.0% w/w tofacitinib, from about 2% w/w to about 10% w/w benzoyl peroxide (BPO), from about 0.01% w/w to about 0.3% w/w tretinoin and a carrier suitable for topical administration.

In some embodiments, there is provided a topical composition for the treatment, prevention or alleviation of an inflammatory skin condition selected from acne and rosacea, comprising from about 0.1% w/w to about 3.0% w/w tofacitinib, from about 2% w/w to about 10% w/w benzoyl peroxide (BPO) and a carrier suitable for topical administration.

In some embodiments, there is provided a topical composition for the treatment, prevention or alleviation of an inflammatory skin condition selected from acne and rosacea, comprising from about 0.1% w/w to about 3.0% w/w tofacitinib, from about 0.01% w/w to about 0.3% w/w tretinoin and a carrier suitable for topical administration.

In some embodiments, there is provided a topical composition for the treatment, prevention or alleviation of an inflammatory skin condition, comprising from about 0.1% w/w to about 3.0% w/w tofacitinib, from about 0.1% w/w to about 2% w/w tapinarof and a carrier suitable for topical administration.

In some embodiments, there is provided a topical composition for the treatment, prevention or alleviation of an inflammatory skin condition selected from acne and rosacea, comprising from about 0.1% w/w to about 3.0% w/w tofacitinib, from about 0.1% w/w to about 2% w/w tapinarof, from about 0.1% w/w to about 3.0% w/w ozenoxacin and a carrier suitable for topical administration. In some embodiments, there is provided a topical composition for the treatment, prevention or alleviation of an inflammatory skin condition, comprising from about 0.1% w/w to about 3.0% w/w tofacitinib, from about 2% w/w to about 10% w/w benzoyl peroxide (BPO), from about. 0.1% w/w to about 3% w/w clascoterone, and a carrier suitable for topical administration.

In some embodiments, there is provided a topical composition for the treatment, prevention or alleviation of an inflammatory skin condition selected from acne and rosacea, comprising from about 0.1% w/w to about 3.0% w/w tofacitinib as the sole active agent.

In some embodiments, there is provided a topical composition for the treatment, prevention or alleviation of an inflammatory skin condition selected from acne and rosacea, comprising from about 0.1% w/w to about 1.0% w/w tofacitinib as the sole active agent.

Tapinarof (3,5-dihydroxy-4-isopropyl-trans-stilbene), benvitimod, GSK2894512 is a first-in-class drug in development, which showed promising results in the topical treatment of psoriasis and also showed activity as acaricide. The acaricide activity of tapinarof is used in this invention in the treatment of rosacea, caused i.a. by the Demodex folliculorum parasite.

Ozenoxacin is a quinolone antibiotic used for the treatment of impetigo.

Benzoyl peroxide (BPO) is marketed for treatment of mild to moderate acne, alone (OTC) or in combination with another active agent (adapalene, clindamycin, erythromycin as topical gel).

Due to its peroxide chemical structure, BPO presents several problems:

a. BPO is a strong oxidant, which may compromise the chemical stability of the other active agents in the combination compositions of this invention and
b. Side-effects like skin irritation, itching, peeling and reddened skin.

The BPO-comprising compositions of this invention use micronized BPO as raw-material, but also several solutions to the above problems:
BPO encapsulation according U.S. Pat. No. 9,687,465 and published U.S. Patent Application No. 2018147165 (to Sol-Gel Technologies), whose contents are enclosed herein in their entirety, thus protecting the at least one additional active agent from the oxidation effect of BPO in a single composition and minimizing the skin irritation side-effect.
Topical application of the BPO-comprising compositions of this invention to the affected skin area of a subject in need thereof as two separate compositions (simultaneously or sequentially in either order) to be rejoined on the subject's skin, the first composition comprising from about 2% to about 10% benzoyl peroxide and a carrier suitable for topical administration and the second composition comprising from about 0.1% to about 3% w/w at least one additional active agent and a carrier suitable for topical administration (see Example 1). Due to this mode of administration, BPO does not compromise the chemical stability of the other active agents in the combination compositions of this invention. The administration can be done for example by applying the two separate compositions to the affected area of the skin of a subject in need thereof from two application syringes or from a dual chamber application syringe, simultaneously or sequentially in either order. In a preferred product according to the invention, the first and second compositions are respectively filled in the chambers of a dual chamber dispensing system of the type described in EP-A-0644129 and U.S. Pat. No. 5,356,040, the contents of which are incorporated herein by reference. Such a system has two side-by-side chambers, each equipped with a dispensing valve; these are operated by adjacent actuators so as to dispense the formulations either simultaneously or separately as desired. Suitable dispensing systems. having chambers which are each capable of holding about 15 ml of composition, are available from Maplast S. r. l., Via Pasublo 3, Tradate 21049 VA, Italy. The respective dimensions of the dispenser means may be chosen to provide dispensing of the respective compositions in a predetermined ratio.

Topical combination compositions have chemical stability problems, caused either by the interaction between the various active agents, or by interaction of the active agents with the carrier.

One of the solutions for this chemical stability problem is the encapsulation of one or more of the active agents in the combination composition. The preferred encapsulation method of this invention is detailed in U.S. Patent No. 9687465 and published U.S. Patent Application No. 2018147165 (to Sol-Gel Technologies), whose contents are enclosed herein in their entirety. Thus, for example, the JAK inhibitor (e.g. tofacitinib) in the compositions of this invention may be encapsulated as disclosed above.

The combination compositions of this invention are useful for the treatment, prevention or alleviation of inflammatory skin conditions, by topical administration to the affected skin area of a subject in need thereof a therapeutically effective amount of said combination compositions. The combination compositions exhibit synergistic and/or additive effects, thus allowing to reduce the amounts of the active agents in the compositions.

The combination of active agents from two or more different classes is more effective than the individual single drugs, with higher clearance rates and/or longer remission periods between clearance and reappearance of the inflammatory skin condition.

### Topical JAK Inhibitor Combination Compositions

Provided herein are compositions, combinations, kits and articles of manufacture for treatment of an inflammatory skin condition, comprising from about 0.1% w/w to about 3.0% w/w at least one JAK inhibitor and at least one additional active agent selected from about 2% w/w to about 10% w/w BPO, from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration.

The active agents of the above compositions belong to seven different active agent classes and may be combined in various ways, providing a large number of possible combinations (see Tables 1 and 2 below). Each combination is a separate embodiment.

### Exemplary Combination Compositions

Several exemplary combination compositions are detailed in Table 1 below. Each of the combinations below is a separate embodiment.

TABLE 1
Exemplary Combinations of Active Agents Classes

| | | | | | | | |
|---|---|---|---|---|---|---|---|
| Active Agent Classes | | | | | | | |
| Combinations | JAKi | BPO | RET | TAP | AA | AC | AB |

| | | | | | | |
|---|---|---|---|---|---|---|
| JAKi/BPO/RET | + | + | + | | | |
| JAKi/BPO | + | + | | | | |
| JAKi/RET | + | | + | | | |
| JAKi/TAP | + | | | + | | |
| JAKi/AB | + | | | | | + |
| JAKi/AA | + | | | | + | |
| JAKi/AA/AB | + | | | | + | + |
| JAKi/BPO/AB | + | + | | | | + |

Legend: JAK Inhibitor (JAKi), Benzoyl peroxide (BPO), Retinoid (RET), Tapinarof (TAP), Antibiotic (AB), Antiandrogen (AA), Acaricide (AC).

Some specific examples of these compositions are described in Table 2 and Examples 1-6.

The compositions, combinations and articles of manufacture of this invention can be administered using a variety of routes such as topical application or transdermal application. The preferred route is the topical route and the preferred formulations are the cream, the lotion, the gel and the foam.

The active agents in the combination compositions are included in an amount effective for treating, preventing or alleviating the inflammatory skin condition or specifically the acne or rosacea symptoms. The concentration of the active agents in the composition will depend on absorption, inactivation, excretion rates of the active agent, the synergistic or additive effects, the dosage schedule, and amount administered as well as other factors known to those of skill in the art.

Typically, the dosages and concentrations of the active agents in the composition of this invention will be lower, typically at least about or at 5 to 10% lower but up to about or at 15, 20, 25, 30, 35, 40, 50, 90 or 95% lower than the amount of same active agents in the marketed single drug currently administered or being developed for the treatment of the skin condition. The dosage and regimen of administration may be determined by dose finding studies, as known in the art.

Exemplary strengths and concentrations of the least one JAK inhibitor in the topical combination compositions are 0.1%, 0.25%, 0.5%, 1%, 2% or 3% w/w. Typical strengths in the topical combination compositions of this invention are 0.1%, 0.25%, 0.5% or 1% w/w. In other embodiments, the concentrations of the least one JAK inhibitor is between 0.1% w/w to about 1% w/w, between 0.5% w/w to about 2% w/w, between 1% w/w to about 3% w/w.

Exemplary strengths and concentrations of BPO in the topical combination compositions comprising BPO are 2%, 3%, 4%, 5%, 6%, 7%, 8%, 9% or 10% w/w. Typical strengths in the topical combination compositions of this invention are 5%, or 10% w/w. In other embodiments, the concentrations of BPO is between 2% w/w to about 6% w/w, between 3% w/w to about 7% w/w, between 3% w/w to about 10% w/w.

Exemplary strengths and concentrations of the least one retinoid in the topical combination compositions are 0.01%, 0.25%, 0.05%, 0.1%, 0.2% or 0.3% w/w. Typical strengths in the topical combination compositions of this invention are 0.05%, or 0.11% w/w. In other embodiments, the concentrations of the least one retinoid is between 0.01% w/w to about 0.1% w/w, between 0.03% w/w to about 0.2% w/w, between 0.05% w/w to about 0.3% w/w.

Exemplary strengths and concentrations of tapinarof in the topical compositions comprising tapinarof are 0.1%, 0.25%, 0.5%, 1%, 2% or 3% w/w. Typical strengths in the topical combination compositions of this invention are 1% or 2% w/w tapinarof. In other embodiments, the concentrations of tapinarof is between 0.1% w/w to about 1% w/w, between 0.5% w/w to about 2% w/w, between 1% w/w to about 3% w/w.

Exemplary strengths and concentrations of the least one antiandrogen in the topical combination compositions are 0.1%, 0.25%, 0.5%, 1%, 2% or 3% w/w. Typical strength in the topical combination compositions of this invention is 1% w/w. In other embodiments, the concentrations of at least one antiandrogen is between 0.1% w/w to about 1% w/w, between 0.5% w/w to about 2% w/w, between 1% w/w to about 3% w/w.

Exemplary strengths and concentrations of the least one antibiotic in the topical combination compositions are 0.1%, 0.25%, 0.5%, 1%, 2% or 3% w/w. Typical strengths in the topical combination compositions of this invention are 1% or 3% w/w. In other embodiments, the concentrations of at least one antibiotic is between 0.1% w/w to about 1% w/w, between 0.5% w/w to about 2% w/w, between 1% w/w to about 3% w/w.

The frequency of administration can be determined empirically.

Exemplary frequencies are once daily, twice daily, weekly, bi-weekly or monthly. Typical administration frequencies of the topical combination compositions of this invention are once daily and twice daily.

Dosage frequencies can be gradually decreased over time and maintained at a steady dose suitable for long-term—six months, 1 year, 5 years, 10 years or more, up to lifelong administration to control the symptoms of the inflammatory skin condition, or specifically of acne or rosacea. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, sebum control products or any other formulation suitable for topical administration. The preferred compositions are the cream, the lotion, the gel and the foam.

Pharmaceutical carriers or vehicles suitable for administration of the compounds provided herein include any such carriers known to those skilled in the art to be suitable for the particular mode of administration.

Sebum control products may include ingredients selected from azelaic acid, salicylic acid, sulfur, nicotinamide, L-carnitine and combinations thereof.

In addition, the compounds may be formulated as the sole pharmaceutically active ingredient in the composition or may be combined with other active agents. The active agents are included in the carrier in an amount sufficient to exert a therapeutically useful effect i.e., amelioration of the symptoms of an inflammatory skin conditions or specifically acne or rosacea, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, foams, solutions, gels, patches and the like. Generally, the vehicle is either organic in nature or an aqueous emulsion and capable of accommodating the selected active agent(s), which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, or specifically from acne or rosacea, by topical administration to a subject in need thereof a therapeutically effective amount of the composition comprising from about 0.1% w/w to about 3.0% w/w at least one JAK inhibitor and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about 0.1% w/w to about 3% w/w at least one antiandrogen, from about 0.5% to about 5% w/w at least one acaricide and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and a pre-filled applicator syringe.

According to an aspect of the invention, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, or specifically from acne or rosacea, by topical administration to a subject in need thereof a therapeutically effective amount of the composition comprising from about 0.1% w/w to about 3.0% w/w tofacitinib as the sole active agent. In another embodiment, from about 0.1% w/w to about 1.0% w/w tofacitinib as the sole active agent.

In some embodiments, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis. In another aspect of this invention, the inflammatory skin condition is acne. In another aspect of this invention, the inflammatory skin condition is rosacea. In another aspect of this invention, the inflammatory skin condition is atopic dermatitis. In another aspect of this invention, the inflammatory skin condition is psoriasis. In another aspect of this invention, the inflammatory skin condition is flexural/inverse psoriasis. In another aspect of this invention, the inflammatory skin condition is eczema. In another aspect of this invention, the inflammatory skin condition is contact dermatitis. In another aspect of this invention, the inflammatory skin condition is urticaria. In another aspect of this invention, the inflammatory skin condition is dermatitis herpetiformis. In another aspect of this invention, the inflammatory skin condition is lichen planus. In another aspect of this invention, the inflammatory skin condition is seborrheic dermatitis.

In some embodiments, the effective amount is a therapeutically effective amount of the active agents, namely an amount which will cure, treat, prevent or alleviate an inflammatory skin condition, or specifically acne or rosacea.

In some embodiments, co-administration of from about 0.1% w/w to about 3.0% w/w at least one JAK inhibitor and at least one additional active agent selected from about 2% w/w to about 10% w/w BPO, from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof, exhibits an additive and/or synergistic effect, which allows reducing the amounts of the active agents in the composition of this invention.

In some other embodiments, the co-administration may be made either by administration of a single combination composition, or alternatively by separate administration of a first composition comprising one of the active agents (e.g. benzoyl peroxide) and a carrier suitable for topical administration and a second composition comprising the other active agent(s) and a carrier suitable for topical administration.

In some embodiments, there is provided a method of treatment, prevention or alleviation of acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof, wherein the at least one Janus kinase inhibitor (JAK inhibitor) and the at least one additional active agent are administered as two or more separate compositions.

In another embodiment, the at least one Janus kinase inhibitor (JAK inhibitor) is formulated in a first composition, and the at least one additional active agent is formulated in a second composition. In another embodiment, the at least one Janus kinase inhibitor (JAK inhibitor) is formulated in a first composition, and the at least one additional active agent is formulated in a second composition or if more than one additional active agent, the additional active agents can be formulated in one composition or each in a separate composition.

In another embodiment, each of the two or more separated compositions are administered once daily or twice daily administration a patient in need thereof until complete remission or according to doctor's orders.

### Regimen of Administration of the Topical Combination Compositions

Therapeutically effective concentrations of active agents in the compositions of this invention for treatment, prevention or amelioration of the symptoms manifested by an inflammatory skin condition or specifically by acne or rosacea are determined by empirical methods known in the art.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually decreased over time and maintained at a steady dose suitable for long-term - six months, 1 year, 5 years, 10 years or more, up to lifelong administration to control the symptoms of the skin condition. For example, dosage administration can begin at from twice a day, to once a day, to two times a week, to once a week, to once every two weeks or less frequent than once every two weeks.

### Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the single composition or two separate compositions to be delivered.

The compositions provided herein can be packaged as articles of manufacture containing packaging material, a composition provided herein, and a label that indicates that the composition is for treating an inflammatory skin disease or specifically acne or rosacea, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes or dual chamber application syringes and any packaging material suitable for the selected formulation and intended mode of administration and treatment.

### Embodiments

In some embodiments, there is provided a topical combination composition comprising from about 0.1% w/w to about 3.0% w/w at least one Janus kinase inhibitor (JAK inhibitor) and at least one additional active agent selected from about 2% w/w to about 10% w/w benzoyl peroxide

(BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration.

In some embodiments, there is provided a composition comprising from about 0.1% w/w to about 3.0% w/w tofacitinib as sole active agent and a carrier suitable for topical administration. In another embodiment from about 0.1% w/w to about 1.0% w/w tofacitinib as sole active agent and a carrier suitable for topical administration.

In some embodiments, there is provided a composition comprising from about 0.1% w/w to about 3.0% w/w JAK inhibitor and from about 0.01% w/w to about 10% w/w of one or two additional active agents selected from BPO, a retinoid, tapinarof, an antibiotic, an acaricide and an antiandrogen, as detailed in Tables 1 and 2.

In some embodiments, there is provided a composition comprising from about 0.1% w/w to about 3.0% w/w at least one JAK inhibitor selected from tofacitinib, abrocitinib delgocitinib and ruxolitinib, from about 2% w/w to about 10% w/w BPO, from about 0.01% w/w to about 0.3% w/w at least one retinoid selected from tretinoin, adapalene and tazarotene and a carrier suitable for topical administration.

In some embodiments, there is provided a composition comprising from about 0.1% w/w to about 3.0% w/w tofacitinib, from about 2% w/w to about 10% w/w BPO from about 0.01% w/w to about 0.3% w/w tretinoin and a carrier suitable for topical administration.

In some embodiments, there is provided a composition comprising from about 0.1% w/w to about 3.0% w/w of a JAK inhibitor selected from tofacitinib, abrocitinib, delgocitinib and ruxolitinib, from about 2% w/w to about 10% w/w BPO and a carrier suitable for topical administration.

In some embodiments, there is provided a composition comprising from about 0.1% w/w to about 3.0% w/w of tofacitinib, from about 2% w/w to about 10% w/w BPO and a carrier suitable for topical administration.

In some embodiments, there is provided a composition comprising from about 0.1% w/w to about 3.0% w/w at least one JAK inhibitor selected from tofacitinib, abrocitinib, delgocitinib and ruxolitinib, from about 0.01% w/w to about 0.3% w/w at least one retinoid selected from tretinoin, adapalene and tazarotene and a carrier suitable for topical administration.

In some embodiments, there is provided a composition comprising from about 0.1% w/w to about 3.0% w/w tofacitinib, from about 0.01% w/w to about 0.3% w/w tretinoin, and a carrier suitable for topical administration.

In some embodiments, there is provided a composition comprising from about 0.1% w/w to about 3.0% w/w at least one JAK inhibitor selected from tofacitinib, abrocitinib, delgocitinib and ruxolitinib, from about 0.1% w/w to about 2% w/w tapinarof and a carrier suitable for topical administration.

In some embodiments, there is provided a composition comprising from about 0.1% w/w to about 3.0% w/w tofacitinib, from about 0.1% w/w to about 2% w/w tapinarof and a carrier suitable for topical administration.

In some embodiments, there is provided a composition comprising from about 0.1% w/w to about 3.0% w/w at least one JAK inhibitor selected from tofacitinib, abrocitinib, delgocitinib and ruxolitinib, from about 0.1% w/w to about 2% w/w at least one antibiotic selected from ozenoxacin, minocycline, doxycycline, clindamycin, clarithromycin and erythromycin and a carrier suitable for topical administration.

In some embodiments, there is provided a composition comprising from about 0.1% w/w to about 3.0% w/w tofacitinib, from about 0.1% w/w to about 3% w/w at least one antibiotic selected from ozenoxacin and minocycline and a carrier suitable for topical administration.

In some embodiments, there is provided a composition comprising from about 0.1% w/w to about 3.0% w/w at least one JAK inhibitor, from about 0.1% w/w to about 3% w/w at least one antiandrogen and a carrier suitable for topical administration. In some embodiments, the at least one antiandrogen is selected from clascoterone, cyproterone, cioteronel and combinations thereof.

In some embodiments, there is provided a composition comprising from about 0.1% w/w to about 3.0% w/w at least one JAK inhibitor selected from tofacitinib, abrocitinib, delgocitinib and ruxolitinib, from about 0.5% w/w to about 5% w/w at least one acaricide selected from ivermectin and permethrin and a carrier suitable for topical administration.

In some embodiments, there is provided a composition comprising from about 0.1% w/w to about 3.0% w/w at least one JAK inhibitor, from about 0.1% w/w to about 3% w/w at least one antiandrogen, from about 0.5% to about 5% w/w at least one acaricide and a carrier suitable for topical administration.

In some embodiments, there is provided a composition comprising from about 0.1% w/w to about 3.0% w/w at least one JAK inhibitor, from about 0.1% w/w to about 3% w/w at least one antiandrogen selected from clascoterone, cyproterone, cioteronel and combinations thereof and a carrier suitable for topical administration.

In some embodiments, there is provided a composition comprising from about 0.1% w/w to about 3.0% w/w at least one JAK inhibitor selected from tofacitinib, abrocitinib, delgocitinib and ruxolitinib, from about 0.5% w/w to about 5% w/w at least one acaricide selected from ivermectin and permethrin and a carrier suitable for topical administration.

In some embodiments, there is provided a composition of this invention, wherein said at least one JAK inhibitor is selected from a JAK1 inhibitor, a JAK2 inhibitor, a JAK3 inhibitor, a TYK2 inhibitor and combinations thereof.

In some embodiments, there is provided any one of the compositions of this invention, wherein said at least one JAK inhibitor is a pan-JAK inhibitor.

In some embodiments, there is provided any one of the methods of treatment of this invention, wherein said at least one JAK inhibitor is a pan-JAK inhibitor.

In some embodiments, there is provided a composition of this invention, wherein said at least one JAK inhibitor is selected from tofacitinib, abrocitinib, delgocitinib, ruxolitinib and combinations thereof.

In some embodiments, there is provided a composition of this invention, wherein said at least one retinoid is selected from tretinoin, adapalene, tazarotene and combinations thereof.

In some embodiments, there is provided a dosage form comprising a composition of this invention, wherein the composition is formulated as a cream, a gel, an ointment, an emulsion, a solution, a suspension, an elixir, a lotion, a tincture, a paste, a foam, an aerosol, a spray, a patch, a transdermal patch, a shampoo and an applicator syringe.

In some embodiments, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof a therapeutically effective amount of a composition comprising from about 0.1% w/w to about 3.0% w/w at least one Janus kinase inhibitor (JAK inhibitor) and optionally at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

In some embodiments, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof a therapeutically effective amount of a composition comprising from about 0.1% w/w to about 3.0% w/w at least one Janus kinase inhibitor (JAK inhibitor) and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

In some embodiments, there is provided a method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis. In another aspect of this invention, the inflammatory skin condition is acne. In another aspect of this invention, the inflammatory skin condition is rosacea. In another aspect of this invention, the inflammatory skin condition is atopic dermatitis. In another aspect of this invention, the inflammatory skin condition is psoriasis. In another aspect of this invention, the inflammatory skin condition is flexural/inverse psoriasis. In another aspect of this invention, the inflammatory skin condition is eczema. In another aspect of this invention, the inflammatory skin condition is contact dermatitis. In another aspect of this invention, the inflammatory skin condition is urticaria. In another aspect of this invention, the inflammatory skin condition is dermatitis herpetiformis. In another aspect of this invention, the inflammatory skin condition is lichen planus. In another aspect of this invention, the inflammatory skin condition is seborrheic dermatitis.

In some embodiments, there is provided a method of treatment, prevention or alleviation of acne, by topical administration to a subject in need thereof a therapeutically effective amount of a composition comprising from about 0.1% w/w to about 3.0% w/w tofacitinib as sole active agent. In another embodiment, from about 0.1% w/w to about 3.0% w/w tofacitinib as sole active agent.

In some embodiments, there is provided a method of treatment, prevention or alleviation of acne, by topical administration to a subject in need thereof a therapeutically effective amount of a composition comprising from about 0.1% w/w to about 1.0% w/w tofacitinib as sole active agent.

In some embodiments, there is provided a method of treatment, prevention or alleviation of an inflammatory skin condition selected from acne and rosacea, by topical administration to a subject in need thereof a therapeutically effective amount of a composition comprising from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 3.0% w/w at least one antibiotic, from about. 0.1% w/w to about 3% w/w at least one antiandrogen and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe, wherein the skin condition is selected from acne and rosacea.

In some embodiments, there is provided a method of treatment, prevention or alleviation of an inflammatory skin condition selected from acne and rosacea, by topical administration to a subject in need thereof a therapeutically effective amount of a composition comprising from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe wherein the skin condition is selected from acne and rosacea, wherein the treatment comprises once daily or twice daily topical administration to a subject in need thereof a therapeutically effective amount of said composition.

In some embodiments, there is provided a method of treatment, prevention or alleviation of an inflammatory skin condition selected from acne and rosacea, by topical administration to a subject in need thereof of a therapeutically effective amount of the composition of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe wherein the skin condition is selected from acne and rosacea, wherein the JAK inhibitor and the at least one additional active agent exhibit an additive or synergistic effect, thereby allowing to reduce the amounts of the active agents in the composition.

In some embodiments, there is provided a method of treatment, prevention or alleviation of acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof, wherein the at least one Janus kinase inhibitor (JAK inhibitor) and the at least one additional active agent are administered as two or more separate compositions wherein the compositions are each formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

In another embodiment, the at least one Janus kinase inhibitor (JAK inhibitor) is formulated in a first composition, and the at least one additional active agent is formulated in a second composition. In another embodiment, the at least one Janus kinase inhibitor (JAK inhibitor) is formulated in a first composition, and the at least one additional active agent is formulated in a second composition or if more than one additional active agent, the additional active agents can be formulated in one composition or each in a separate composition.

In another embodiment, each of the two or more separated compositions are administered once daily or twice daily administration a patient in need thereof until complete remission or according to doctor's orders.

In some embodiments there is provided a regimen of administration comprising the once daily or twice daily administration a patient in need thereof of a therapeutically effective amount of a dosage form comprising a composition comprising from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration until complete remission or according to doctor's orders.

In some embodiments there is provided a kit comprising instructions for use and one or more dosage forms comprising a composition comprising from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof and a carrier suitable for topical administration.

JAK inhibitors may be used for the topical or oral treatment of inflammatory skin conditions or specifically acne and rosacea also as sole active agents(s), that is without any additional active agents.

In some embodiments, there is provided a method of treatment, prevention or alleviation of an inflammatory skin condition selected from acne and rosacea, by administration to a subject in need thereof a therapeutically effective amount of a topical composition comprising from about 0.1% w/w to about 3.0% w/w at least one JAK inhibitor or an oral composition comprising from about 1 mg to about 25 mg or from about 25 mg to about 50 mg at least one JAK inhibitor.

In some embodiments, there is provided a method of treatment, prevention or alleviation of an inflammatory skin condition selected from acne and rosacea, by administration to a subject in need thereof a therapeutically effective amount of a topical composition comprising from about 0.1% w/w to about 3.0% w/w at least one JAK inhibitor or an oral composition comprising from about 1 mg to about 25 mg or from about 25 mg to about 50 mg at least one JAK inhibitor, wherein the at least one JAK inhibitor is selected from tofacitinib, abrocitinib, delgocitinib, ruxolitinib and combinations thereof.

In some embodiments, there is provided a method of treatment, prevention or alleviation of an inflammatory skin condition selected from acne and rosacea, by topical administration to a subject in need thereof a therapeutically effective amount of a composition comprising from about 0.1% w/w to about 3.0% w/w at least one Janus kinase inhibitor (JAK inhibitor) as sole active agent and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

In some embodiments, there is provided a method of treatment, prevention or alleviation of an inflammatory skin condition selected from acne and rosacea, by oral administration to a subject in need thereof a therapeutically effective amount of an oral composition comprising from about 1 mg to about 25 mg or from about 25 mg to about 50 mg at least one JAK inhibitor as sole active agent and excipients suitable for oral administration, wherein the composition is formulated in a dosage form selected from a tablet, a capsule, a sachet, a powder, a syrup, a solution, an oral film, an oral dosage delivery system and oral granules.

In some embodiments, there is provided any one of the aforementioned methods of treatment of this invention, wherein the treatment comprises once daily or twice daily oral administration to a subject in need thereof a therapeutically effective amount of said oral composition.

In some embodiments, there is provided a method of treatment, prevention or alleviation of an inflammatory skin condition selected from acne and rosacea, by topical administration to a subject in need thereof a therapeutically effective amount of a composition comprising from about 0.1% w/w to about 3.0% w/w at least one Janus kinase inhibitor (JAK inhibitor) selected from tofacitinib, abrocitinib, delgocitinib and ruxolitinib as sole active agent and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “agent” or “active agent” or “active pharmaceutical ingredient” or “API” are interchangeable and mean the ingredient is a pharmaceutical active agent which is biological active and is regulatory approved or approvable as such.

As used herein, the terms “inflammatory skin conditions”, “inflammatory skin disorders”, “inflammatory skin diseases” and “inflammatory cutaneous conditions” are any medical conditions affecting the integumentary system and are used interchangeably.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Table 2 below details some exemplary combinations of specific active agents of this invention:

TABLE 2
Exemplary Combinations of Specific Active Agents

| | | | | | | | |
|---|---|---|---|---|---|---|---|
| Active Agent Classes | | | | | | | |
| Actives' Combinations | JAKi | BPO | RET | TAP | AA | AC | AB |

| | | | | | |
|---|---|---|---|---|---|
| TOFA/BPO/TRET | + | + | + | | |
| TOFA/BPO | + | + | | | |
| TOFA/TRET | + | | + | | |
| TOFA/TAP | + | | | + | |
| TOFA/IVR | + | | | | |
| TOFA/TAP/OZNX | + | | | + | |
| TOFA/BPO/CLAS | + | + | | | + |

Legend: JAK Inhibitor (JAKi), Benzoyl peroxide (BPO), Retinoid (RET), Tapinarof (TAP), Antibiotic (AB), Antiandrogen (AA), Acaricide (AC) Tofacitinib (TOFA), Ivermectin

(IVR)Tretinoin (TRET), Ozenoxacin (OZNX), Clascoterone (CLAS).

Generally, the nomenclature used herein and the laboratory procedures utilized in the present invention include chemical, molecular and biochemical, techniques. Such techniques are thoroughly explained in the literature. General references are provided throughout this document.

The procedures therein are believed to be well known in the art and are provided for the convenience of the reader. All the information contained therein is incorporated herein by reference.

### Example 1

### Preparation of a Tofacitinib-BPO-Tretinoin Topical Combination

The topical tofacitinib-BPO-tretinoin combination consists of two separate compositions, to be administered to the affected skin area of a subject in need thereof as two separate compositions from two application syringes or a dual chamber application syringe, simultaneously or sequentially in either order.

### Composition 1: Preparation of the Tofacitinib/Tretinoin Composition

0.2%-6.0% w/w tofacitinib,
0.02%-0.6% w/w tretinoin,
4%-5% w/w cyclomethicone,
21%-27% coconut oil,
46%-54% w/w soybean oil,
2%-3% miristyl alcohol,
1%-1.5% w/w behenyl alcohol,
3.0%-20% w/w cetyl alcohol.

Composition 1 is prepared by the following steps:

Weigh in a beaker cyclomethicone (24 g), coconut oil (123 g), soybean oil (254.5 g), myristyl alcohol (12.5 g), cetyl alcohol (35 g) and behenyl alcohol (5.5 g). Transfer the obtained mixture to a 70° C. water bath and mix with a magnetic stirrer until the ingredients are fully dissolved. After the full dissolution, reduce the bath temperature to 50° C.

Weigh tofacitinib (30 g) and tretinoin (3 g) in a weighing dish and add to the above solution of the other ingredients in the beaker at 50° C. while stirring at high shear (7000 rpm) during 5 minutes.

Transfer the contents of the beaker to an ice-cooled water bath and cool to room-temperature under manual mixing, thus obtaining about 500 g of composition.

Fill a 15 g portion of the above composition 1 into a topical application syringe or in one of the chambers of a dual chamber topical application syringe.

### Composition 2: Preparation of BPO 5% Cream

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate were mixed at 70° C.

Water phase: 18.0 grams of ethylenediamine tetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 14304 grams of benzoyl peroxide (BPO) 15% water suspension made as described below were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms.

Fill a 15 g portion of the above 5% BPO composition 2 into a topical application syringe or in one of the chambers of a dual chamber topical application syringe.

Preparation of 15% BPO Water Suspension

A benzoyl peroxide (BPO) suspension was prepared by mixing 125.67 grams of CTAC CT-429 (Cetrimonium Chloride 30%), 3000 grams of hydrous benzoyl peroxide, and 5200 g water under high shear. The suspension was homogenized for 60 minutes at 33° C. (no more than 45° C.), and then the pH of the solution was adjusted to 7.0 using sodium hydroxide solution (20%). An acid cocktail was prepared using 493 grams hydrochloric acid (37%), 98 grams anhydrous Citric Acid, 147 grams Lactic Acid (90%), and 794 grams water.

Stabilization of the 15% BPO Water Suspension

Stabilization of the suspension was done by adding 532 grams of silicon dioxide to the benzoyl peroxide suspension prepared in the above step under high shear, followed by adding 855 grams 10% PDAC (Polyquarternium-7) solution to the mixture. The mixture was stirred under high shear for 2 hours. The pH of the mixture was adjusted to 5.0 using the above acid cocktail, and water was added to complete the total weight of the mixture to 15 kilograms.

### Example 2

### Preparation of a Tofacitinib-BPO Topical Combination (Prophetical)

The topical tofacitinib-BPO combination consists of two separate compositions, to be administered to the affected skin area of a subject in need thereof as two separate compositions. from two application syringes or a dual chamber application syringe, simultaneously or sequentially in either order.

Composition 1: Preparation of the tofacitinib composition 1
0.2%-6.0% w/w tofacitinib,
4%-5% w/w cyclomethicone,
21%-27% coconut oil,
46%-54% w/w soybean oil,
2%-3% miristyl alcohol,
1%-1.5% w/w behenyl alcohol,
3.0%-20% w/w cetyl alcohol.

Composition 1 is prepared by the following steps:

Weigh in a beaker cyclomethicone (24 g), coconut oil (123 g), soybean oil (254.5 g), myristyl alcohol (12.5 g), cetyl alcohol (35 g) and behenyl alcohol (5.5 g). Transfer the obtained mixture to a 70° C. water bath and mix with a magnetic stirrer until the ingredients are fully dissolved. After the full dissolution, reduce the bath temperature to 50° C.

Weigh tofacitinib (30 g) in a weighing dish and add to the above solution of the other ingredients in the beaker at 50° C. while stirring at high shear (7000 rpm) during 5 minutes.

Transfer the contents of the beaker to an ice-cooled water bath and cool to room-temperature under manual mixing, thus obtaining about 500 g of composition.

Fill a 15 g portion of the above composition 1 into a topical application syringe or in one of the chambers of a dual chamber topical application syringe.

### Composition 2: Preparation of BPO 5% Cream

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate were mixed at 70° C.

Water phase: 18.0 grams of ethylenediamine tetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 14304 grams of benzoyl peroxide (BPO) 15% water suspension made as described below were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms.

Fill a 15 g portion of the above 5% BPO composition 2 into a topical application syringe or in one of the chambers of a dual chamber topical application syringe.

Preparation of 15% BPO Water Suspension

A benzoyl peroxide (BPO) suspension was prepared by mixing 125.67 grams of CTAC CT-429 (Cetrimonium Chloride 30%), 3008 grams of hydrous benzoyl peroxide, and 5200 g water under high shear. The suspension was homogenized for 60 minutes at 33° C. (no more than 45° C.), and then the pH of the solution was adjusted to 7.0 using sodium hydroxide solution (20%). An acid cocktail was prepared using 493 grams hydrochloric acid (37%), 98 grams anhydrous Citric Acid, 147 grams Lactic Acid (90%), and 794 grams water.

Stabilization of the 15% BPO Water Suspension

Stabilization of the suspension was done by adding 532 grams of silicon dioxide to the benzoyl peroxide suspension prepared in the above step under high shear, followed by adding 855 grams 10% PDAC (Polyquarternium-7) solution to the mixture. The mixture was stirred under high shear for 2 hours. The pH of the mixture was adjusted to 5.0 using the above acid cocktail, and water was added to complete the total weight of the mixture to 15 kilograms.

### Example 3

### Preparation of a Tofacitinib-Tretinoin Topical Combination Composition

The topical tofacitinib-tretinoin combination composition consists of:

0.1%-3.0% w/w tofacitinib,
0.01%-0.3% w/w tretinoin,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 0.1M NaOH or 0.1M HCl.

The composition is prepared by the following steps:

(1) weigh tofacitinib and tretinoin having an average particle size of less than 1 μm;
(2) heat the propylene glycol to 60° C. in a water bath;
(3) add to the heated propylene glycol while stirring tofacitinib, tretinoin, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80, make up to 100% with purified water and adjust the pH to 6.0-7.0 with 0.1 M NaOH or 0.1M HCl;
(5) add the aqueous phase to the oil phase under vacuum stirring, and cool to room temperature to obtain a cream;
(6) fill the tofacitinib-tretinoin combination composition in tubes or other delivery system.

### Example 4

### Preparation of a Tofacitinib-Tapinarof Topical Combination Composition

The topical tofacitinib-tapinarof combination composition consists of:

0.1%-3.0% w/w tofacitinib,
0.1%-2% w/w tapinarof,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 0.1M NaOH or 0.1M HCl,

The composition is prepared by the following steps:

(1) weigh tofacitinib and tapinarof having an average particle size of less than 1 μum;
(2) heat the propylene glycol to 60° C. in a water bath;
(3) add to the heated propylene glycol while stirring tofacitinib, tretinoin, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80, make up to 100% with purified water and adjust the pH to 6.0-7.0 with 0.1 M NaOH or 0.1M HCl;
(5) add the aqueous phase to the oil phase under vacuum stirring, and cool to room temperature to obtain a cream;
(6) fill the tofacitinib-tapinarof combination composition in tubes or other delivery system.

### Example 5

### Preparation of a Tofacitinib-Tapinarof-Ozenoxacin Topical Combination Composition

The topical tofacitinib-tapinarof-ozenoxacin combination composition consists of:

0.1%-3.0% w/w tofacitinib,
0.1%-2.0% w/w tapinarof,
0.1-3.0% w/w ozenoxacin,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 1M NaOH or 0.1M HCl,

The composition is prepared by the following steps:

(1) weigh tofacitinib, ozenoxacin and tapinarof having an average particle size of less than 1 μm;
(2) heat the propylene glycol to 60° C. in a water bath;
(3) add to the heated propylene glycol while stirring tofacitinib, tretinoin, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80, make up to 100% with purified water and adjust the pH to 6.0-7.0 with 0.1 M NaOH or 0.1M HCl;
(5) add the aqueous phase to the oil phase under vacuum stirring, and cool to room temperature to obtain a cream;
(6) fill the tofacitinib-tapinarof-ozenoxacin combination composition in tubes or other delivery system.

### Example 6

### Preparation of a Tofacitinib-BPO-Clascoterone Topical Combination

The topical tofacitinib-BPO-clascoterone combination composition consists of two separate compositions, to be administered to the affected skin area of a subject in need thereof as two separate compositions. from two application syringes or a dual chamber application syringe, simultaneously or sequentially in either order.

Composition 1: Preparation of the tofacitinib-clascoterone composition 1
0.2%-6. 0% w/w tofacitinib,
0.2%-6% w/w clascoterone,
4%-5% w/w cyclomethicone,
21%-27% coconut oil,
46%-54% w/w soybean oil,
2%-3% miristyl alcohol,
1%-1.5% w/w behenyl alcohol,
3.0%-20% w/w cetyl alcohol.

Composition 1 is prepared by the following steps:

Weigh in a beaker cyclomethicone (24 g), coconut oil (123 g), soybean oil (254.5 g), myristyl alcohol (12.5 g), cetyl alcohol (35 g) and behenyl alcohol (5.5 g).Transfer the obtained mixture to a 70° C. water bath and mix with a magnetic stirrer until the ingredients are fully dissolved. After the full dissolution, reduce the bath temperature to 50° C.

Weigh tofacitinib (30 g) and clascoterone (30 g) in a weighing dish and add to the above solution of the other ingredients in the beaker at 50° C. while stirring at high shear (7000 rpm) during 5 minutes.

Transfer the contents of the beaker to an ice-cooled water bath and cool to room-temperature under manual mixing, thus obtaining about 500 g of composition.

Fill a 15 g portion of the above composition 1 into a topical application syringe or in one of the chambers of a dual chamber topical application syringe.

### Composition 2: Preparation of BPO 5% Cream

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate were mixed at 70° C.

Water phase: 18.0 grams of ethylenediamine tetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 6,000 grams of benzoyl peroxide (BPO) 15% water suspension made as described below were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms.

Fill a 15 g portion of the above 5% BPO composition 2 into a topical application syringe or in one of the chambers of a dual chamber topical application syringe.

Preparation of 15% BPO Water Suspension

A benzoyl peroxide (BPO) suspension was prepared by mixing 125.67 grams of CTAC CT-429 (Cetrimonium Chloride 30%), 3008 grams of hydrous benzoyl peroxide, and 5200 g water under high shear. The suspension was homogenized for 60 minutes at 33° C. (no more than 45° C.), and then the pH of the solution was adjusted to 7.0 using sodium hydroxide solution (20%). An acid cocktail was prepared using 493 grams hydrochloric acid (37%), 98 grams anhydrous Citric Acid, 147 grams Lactic Acid (90%), and 794 grams water.

Stabilization of the 15% BPO Water Suspension

Stabilization of the suspension was done by adding 532 grams of silicon dioxide to the benzoyl peroxide suspension prepared in the above step under high shear, followed by adding 855 grams 10% PDAC (Polyquarternium-7) solution to the mixture. The mixture was stirred under high shear for 2 hours. The pH of the mixture was adjusted to 5.0 using the above acid cocktail, and water was added to complete the total weight of the mixture to 15 kilograms.

1. A topical composition comprising from about 0.1% w/w to about 3.0% w/w at least one Janus kinase inhibitor (JAK inhibitor) and optionally at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about.

0. 1% w/w to about 3% w/w at least one antiandrogen, from about 0.5% to about 5% w/w at least one acaricide and combinations thereof and a carrier suitable for topical administration.

2. The composition of claim 1, wherein the at least one JAK inhibitor from about 0.1% w/w to about 3.0% w/w is tofacitinib as sole active agent.

3. The composition of claim 1, wherein the at least one JAK inhibitor from about 0.1% w/w to about 1.0% w/w is tofacitinib as sole active agent.

4. The composition of claim 1, comprising from about 0.1% w/w to about 3.0% w/w JAK inhibitor and from about 0.01% w/w to about 10% w/w of one or two additional active agents selected from BPO, a retinoid, tapinarof, an antibiotic, an acaricide and an antiandrogen, as detailed in Tables 1 and 2.

5. The composition of claim 1, comprising from about 0.1% w/w to about 3.0% w/w at least one JAK inhibitor selected from tofacitinib, abrocitinib, delgocitinib and ruxolitinib, from about 2% w/w to about 10% w/w BPO, from about 0.01% w/w to about 0.3% w/w at least one retinoid selected from tretinoin, adapalene and tazarotene and a carrier suitable for topical administration.

6. The composition of claim 4, comprising from about 0.1% w/w to about 3.0% w/w tofacitinib, from about 2% w/w to about 10% w/w BPO from about 0.01% w/w to about 0.3% w/w tretinoin and a carrier suitable for topical administration.

7. The composition of claim 1, comprising from about 0.1% w/w to about 3.0% w/w of a JAK inhibitor selected from tofacitinib, abrocitinib, delgocitinib and ruxolitinib, from about 2% w/w to about 10% w/w BPO and a carrier suitable for topical administration.

8. The composition of claim 7, comprising from about 0.1% w/w to about 3.0% w/w of tofacitinib, from about 2% w/w to about 10% w/w BPO and a carrier suitable for topical administration.

9. The composition of claim 1, comprising from about 0.1% w/w to about 3.0% w/w at least one JAK inhibitor selected from tofacitinib, abrocitinib, delgocitinib and ruxolitinib, from about 0.01% w/w to about 0.3% w/w at least one retinoid selected from tretinoin, adapalene and tazarotene and a carrier suitable for topical administration.

10. The composition of claim 9, comprising from about 0.1% w/w to about 3.0% w/w tofacitinib, from about 0.01% w/w to about 0.3% w/w tretinoin, and a carrier suitable for topical administration.

11. The composition of claim 1, comprising from about 0.1% w/w to about 3.0% w/w at least one JAK inhibitor selected from tofacitinib, abrocitinib, delgocitinib and ruxolitinib, from about 0.1% w/w to about 2% w/w tapinarof and a carrier suitable for topical administration.

12. The composition of claim 11, comprising from about 0.1% w/w to about 3.0% w/w tofacitinib, from about 0.1% w/w to about 2% w/w tapinarof and a carrier suitable for topical administration.

13. The composition of claim 1, comprising from about 0.1% w/w to about 3.0% w/w at least one JAK inhibitor selected from tofacitinib, abrocitinib, delgocitinib and ruxolitinib, from about 0.1% w/w to about 2% w/w at least one antibiotic selected from ozenoxacin, minocycline, doxycycline, clindamycin, clarithromycin and erythromycin and a carrier suitable for topical administration.

14. The composition of claim 13, comprising from about 0.1% w/w to about 3.0% w/w tofacitinib, from about 0.1% w/w to about 3% w/w at least one antibiotic selected from ozenoxacin and minocycline and a carrier suitable for topical administration.

15. The composition of claim 1, comprising from about 0.1% w/w to about 3.0% w/w at least one JAK inhibitor, from about 0.1% w/w to about 3% w/w at least one antiandrogen and a carrier suitable for topical administration.

16. The composition of claim 1, wherein the at least one antiandrogen is selected from clascoterone, cyproterone, cioteronel and combinations thereof.

17. The composition of claim 1, comprising from about 0.1% w/w to about 3.0% w/w at least one JAK inhibitor selected from tofacitinib, abrocitinib, delgocitinib and ruxolitinib, from about 0.5% w/w to about 5% w/w at least one acaricide selected from ivermectin and permethrin and a carrier suitable for topical administration.

18. The composition of claim 1, wherein the at least one JAK inhibitor is selected from a JAK1 inhibitor, a JAK2 inhibitor, a JAK3 inhibitor, a TYK2 inhibitor, a pan-JAK inhibitor and combinations thereof.

19. The composition of claim 1, wherein the at least one JAK inhibitor is a pan-JAK inhibitor.

20. The composition of claim 1, wherein the at least one JAK inhibitor is selected from tofacitinib, abrocitinib, delgocitinib, ruxolitinib and combinations thereof.

21. The composition of claim 1, wherein the at least one retinoid is selected from tretinoin, adapalene, tazarotene and combinations thereof.

22. A dosage form comprising the composition of claim 1, wherein the composition is formulated as a cream, a gel, an ointment, an emulsion, a solution, a suspension, an elixir, a lotion, a tincture, a paste, a foam, an aerosol, a spray, a patch, a transdermal patch and an applicator syringe.

23. A method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof a therapeutically effective amount of the composition of claim 1, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

24. The method of claim 23, wherein said skin condition is acne and said composition comprises from about 0.1% w/w to about 3.0% w/w tofacitinib as sole active agent.

25. The method of claim 24, wherein said skin condition is acne and wherein said composition comprising from about 0.1% w/w to about 1.0% w/w tofacitinib as sole active agent.

26. The method of claim 23, wherein the skin condition is selected from acne and rosacea.

27. A method of treatment, prevention or alleviation of an inflammatory skin condition selected from acne and rosacea, by administration to a subject in need thereof a therapeutically effective amount of a topical composition comprising from about 0.1% w/w to about 3.0% w/w at least one JAK inhibitor, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe, or an oral composition.

28. The method of claim 27, wherein the composition comprises from about 1 mg to about 25 mg or from about 25 mg to about 50 mg at least one JAK inhibitor, wherein the oral composition is formulated in a dosage form selected from a tablet, a capsule, a sachet, a powder, a syrup, a solution, an oral film, an oral dosage delivery system and oral granules.

29. The method of claim 23, wherein the at least one JAK inhibitor is selected from tofacitinib, abrocitinib, delgocitinib, ruxolitinib and combinations thereof.

30. The method of claim 23, wherein the treatment comprises once daily or twice daily topical administration to a subject in need thereof a therapeutically effective amount of said composition.

31. The method of claim 27, wherein the treatment comprises once daily or twice daily oral administration to a subject in need thereof a therapeutically effective amount of said oral composition.

32. The method of claim 23, wherein the JAK inhibitor and the at least one additional active agent exhibit an additive or synergistic effect, thereby allowing to reduce the amounts of the active agents in the composition.

33. A method of treatment, prevention or alleviation of an inflammatory skin condition selected from acne, rosacea, atopic dermatitis, psoriasis, flexural/inverse psoriasis, eczema, contact dermatitis, urticaria, dermatitis herpetiformis, lichen planus and seborrheic dermatitis, by topical administration to a subject in need thereof from about 0.1% w/w to about 3.0% w/w at least one Janus kinase inhibitor (JAK inhibitor) and at least one additional active agent selected from about 2% w/w to about 10% w/w benzoyl peroxide (BPO), from about 0.01% w/w to about 0.3% w/w at least one retinoid, from about 0.1% w/w to about 2% w/w tapinarof, from about 0.1% w/w to about 3.0% w/w at least one antibiotic, from about. 0.1% w/w to about 3% w/w at least one antiandrogen, from about 0.5% to about 5% w/w at least one acaricide and combinations thereof, wherein the at least one Janus kinase inhibitor (JAK inhibitor) and the at least one additional active agent are administered as two or more separate compositions wherein the compositions are each formulated in a dosage form selected from a cream, a gel, an ointment, an emulsion, a solution, a suspension, an elixir, a lotion, a tincture, a paste, a foam, an aerosol, a spray, a patch, a transdermal patch, a shampoo and a pre-filled applicator syringe.

34. A regimen of administration comprising the once daily or twice daily administration a patient in need thereof of a therapeutically effective amount of the dosage form of claim 22 until complete remission or according to doctor's orders.

35. A kit comprising one or more dosage forms of claim 22 and instructions for use.
